(19) United States
(12) Patent Application Publication
(10) Pub. No.: US 2017/0314582 A1
(43) Pub. Date: Nov. 2, 2017

(54) PYROTECHNIC DRIVE DEVICE

(71) Applicant: Peter Lell, Moosburg (DE)

(72) Inventor: Peter Lell, Moosburg (DE)

(21) Appl. No.: 15/520,599

(22) PCT Filed: Oct. 21, 2015
(86) PCT No.: PCT/DE2015/100438

§ 371 Date: Apr. 20, 2017

(30) Foreign Application Priority Data
Oct. 22, 2014 (DE) 10 2014 115 397.9

(51) Int. Cl. F15B 15/19; H01H 39/00; F15B 15/14; A61M 5/30; F15B 15/10; C06D 5/02; F15B 15/24

(52) U.S. Cl. CPC F15B 15/19; H01H 39/006; H01H 39/002; F15B 15/24; A61M 5/3015; F15B 15/10; C06D 5/02; F15B 15/14

(57) ABSTRACT

A pyrotechnic drive device includes a housing (3) provided with a combustion chamber (5) having pyrotechnic material (15) as well as an activation device (13). The combustion chamber is delimited at least in an initial state on all sides by combustion chamber walls (3, 7, 13, 17, 33, 35) formed in at least one partial region by respective pressure-receiving surface or respective pressure-receiving element (17, 35). Each pressure-receiving impact of a pressure-receiving element (17, 35) is impacted after the activation of the pyrotechnic material (15) in such a way by the gas pressure generated in this manner that the pressure-receiving element (17, 35) is moved and/or deformed (17, 35) and/or a mechanical impulse is thus transmitted via the pressure-receiving element (17, 35) to an element to be driven (19) so that a connected substance (25) is transmitted. The residual volume of the combustion chamber (5), in which no pyrotechnic material (15) is provided in the initial state, is filled with a liquid, a gel-like or pasty filling material (21), and/or a soft, rubber-like material.

The invention relates to a pyrotechnic drive device provided with the features of the preamble of claim 1.

Pyrotechnic drive devices are used for a variety of purposes, for example as drives for electrical switches, in particular isolating switches, such as those that are used in automotive technology in order to rapidly and permanently separate the battery or accumulator of a motor vehicle from the electrical system of the vehicle in case of danger. Such a disconnecting switch, which can be also referred to as a fuse, is described in WO 03/067621 A1. In one of the variants, a piston-like element is moved when a pyrotechnic material is triggered, wherein a fuse, which is arranged between the movable element and a stationary element, is severed and the current flowing through the current path is thus interrupted. Although in this document, the pyrotechnic drive of the piston-like element is still integrated in the electrical switch, such a drive can of course also be designed as an independent device.

Another application is found in the medical field. Here, it is known in particular that a dust-like or powder-like substance, such as for example a medication, can be accelerated by means of a pyrotechnic drive in such a way that it can be injected directly, which is to say without a carrier liquid and faultlessly, into a tissue. The dust-like or powder-like substance particles are thereby shot directly into the tissue, in particular into the upper layers of the skin of a body, which is a process that is virtually free of pain. Such a device is described for example in the EP 1 599 242 B1. Here, a variant is used which functions by means of a shock wave wherein a pyrotechnic charge acts on a membrane so that a mechanical impulse is transmitted onto the dust-like or powder-like substance adhering to the membrane. The substance particles are thus dissolved and accelerated to a very high speed. In another variant, a piston is accelerated with a pyrotechnic propulsion charge, so that the piston impacts the rear side of the membrane with a correspondingly higher speed and thus transfers a sufficiently high speed via the membrane to the substance particles adhering to it.

With known pyrotechnic drives, whether they are provided integrated in any kind of device or as an independent device, the pyrotechnic material, which is provided to generate the pressure or pressure surge (hereinafter also referred to as shock wave), is introduced into a combustion chamber. The volume of the combustion chamber in this case usually corresponds to the volume that is required for the pyrotechnic material. However, if, depending on the mobility or the burning rate of the pyrotechnical material, only a small amount of the pyrotechnical material is required, or if as little as possible of the pyrotechnic material is to be contained or should be contained in the module for reasons of a higher level of safety in the event of an accident, the problem that is often encountered is that the combustion chamber cannot be formed small enough, or that the pyrotechnic material, which is often present in solid form, for example compressed form, cannot be produced with the tolerance required in order to fill the entire combustion chamber. The residual volume of the combustion chamber, which is not claimed by the pyrotechnic material, and the air present or the gas present in it limits the gradient of the pressure rise, which is generated after the activation of the pyrotechnic material and requires additional energy that is not used by the actual acceleration process of the membrane or of the piston and that also attenuates the shock wave. This reduces the transmission of a rapid mechanical impulse to the output element of the pyrotechnic drive device (hereinafter referred to also as pressure-receiving element).

It should be pointed out at this point that within the context of this description, material characterized by a deflagrating or detonative conversion is referred to as pyrotechnic material. This includes also deflagrating material mixtures such as for example thermite mixtures or tetrazene. A material with a deflagrating reaction generates in this manner a pressure increase or a pressure wave whose propagation velocity is lower than or equal to the sound velocity in the medium in question. A detonatively converting material, on the other hand, produces a pressure change that is referred to as a pressure surge or pressure wave in the medium in question, whose velocity is higher than the sound velocity in the medium.

This essentially results in two different types of pyrotechnic drive devices.

When a deflagrating pyrotechnic material, which is to say relatively slowly reacting pyrotechnical material, is used, this results in a relatively slow pressure change or pressure wave in the millisecond range. If this relatively “slow” pressure increase impacts the pressure-receiving element, the element will undergoes deformation or it will be moved. Both effects on the pressure-receiving element are also possible. Such a relatively slow pressure increase is generally utilized to cause an increase of the combustion chamber volume. For example, a pressure-receiving element designed as a piston can be driven in this manner, or a pressure-receiving element designed as a deformable membrane can be slowly deformed, which leads to an increase of the volume of the combustion chamber and thus also to the desired activation of the membrane surface.

When a detonatively converting pyrotechnic material is used, the generated pressure surge, or the shock wave produced by it, is utilized to generate the output power of the pyrotechnic output device. The pressure surge (the shock wave) reaches the pressure-receiving element or its impacted surface before this can lead to a potential, much slower pressure increase in the combustion chamber volume and result in its increase. The pressure surge can be used to transfer a mechanical output power through the pressure-receiving element to one or to a plurality of elements to be driven, for example to substance particles of a pharmaceutical substance that adhere to a surface of the pressure-receiving element. In this manner, the substance particles are accelerated within a very short time period to a high speed and after they reach the maximum speed, they are dissipated. When the substance particles encounter a technical surface, a tissue or skin, they can penetrate them up to a certain depth as a result of their high energy.

The inventor has recognized that in both cases, a combustion chamber volume that is not completely filled with pyrotechnic material exerts an adverse effect on the mechanical performance of the drive. In the case when a material with a deflagrating conversion is used, the use of an excessive amount of pyrotechnical material (which is to without an unfilled residual volume) is in fact required. In the case of a material with a detonative conversion, the non-filled residual volume of the combustion chamber leads to an attenuation of the shock wave as a result of the material transition between the residual volume and the chamber-side surface of the pressure-receiving element (impacted surface), which leads to a reflection of the shock wave and thus to a decrease of the drive power.

The object of the invention is therefore to provide a pyrotechnic drive device which generates the highest possible and fastest acting drive power with as little pyrotechnic material as possible.

The invention achieves this object with the features of the patent claim 1.

The invention is based on the finding that it is possible to reduce or avoid the negative influence of a residual volume of the combustion chamber, which can be present in the initial state of a pyrotechnic drive device, (which is to say before the activation of the pyrotechnic material), when the residual volume in the initial state of the pyrotechnic drive device (in particular with the pressure and the temperature prevailing in the combustion chamber during the initial state), when it is essentially completely filled with a liquid, a gel-like, or a past-like filling material and/or a soft rubber-like filling material. This is because with the filling material, good coupling of the at least one charging element with the energy released by the pyrotechnic material is achieved and the resulting increased pressure is thus also achieved in the combustion chamber.

The filling material should preferably be in the case of a detonatively converting pyrotechnic material designed or provided in such a way that the shock wave generated with the pyrotechnic material, which has a higher propagation velocity when compared to the sound velocity in the material in question, results in a reflection that is as much free of attenuation as possible and thus has a reflection that is also as small as possible (in particular on the impacted surface of the pressure-receiving element that limits the combustion chamber), when it is transferred through the filling material to the pressure-receiving element.

On the other hand, if a pyrotechnic material with a deflagrating reaction is used, which generates a pressure wave that is slower than the sound velocity of the material in question, the filling material should be preferably designed or provided in such a way that it can respond well to the deformation of the combustion chamber, in particular deformation caused by movement and/or deformation of the at least one pressure-receiving element, so that as little energy as possible will be required for this purpose.

When the residual volume of the combustion chamber is filled with a filling material, the result of the use of a material with a deflagrating reaction is that the reaction process with the gas (hot gas due to conversion of the of the pyrotechnic material by burning) generated in the combustion chamber, an immediate and relatively steep pressure increase is caused, with which the pressure-receiving surface of the at least one pressure-receiving element is impacted. Since the residual volume in the initial state of the drive device does not contain any compressible gas, no energy is extracted, which would be required for the compression of this kind of a residual volume and which could not be converted into drive energy. In the case when a material with a detonative conversion is used, a much lower attenuation or reflection of the shock wave is created during the transfer to the pressure-receiving element.

As a result, the quantity or the mass of the used pyrotechnic material can be significantly reduced when compared to known embodiment forms, in which the combustion chamber is filled with a residual volume in the initial state.

The tolerances required to create the geometry of the combustion chamber and/or during the manufacturing of pyrotechnic materials, for example when creating the geometry of a detonator consisting of an igniting device with a pyrotechnic material attached to it, (this material can be compressed to a solid body and/or connected with a cover to it), can be compensated for with the introduction of a liquid, a gel-like, or a paste-like filing material and/or of a rubber-like filling material into the residual volume, without resulting in the disadvantages described above that are known to occur with pyrotechnic drive devices.

Under liquid, a gel-like or a paste-like filling material is hereinafter to be understood any material that exhibits a liquid, gel-like or paste-like consistency when it is used in an (ambient) working temperature range in which the pyrotechnic drive device may be employed in the resulting state in the combustion chamber (in particular with respect to pressure and temperature). In particular, all natural and mineral oils and carbon-based oils should be included in this case, but also silicon-based oils. Examples of liquid filling materials are rape seed and sunflower oil, but also transformer oil, mineral oil and silicon oils that can be obtained with diverse viscosities and thickening agents such as silicon dioxide, highly dispersed silicas (HDS), glass beads, etc. It goes without saying that different starting materials can be also mixed into the filling materials.

These types of liquids, gel-like or pasty filling materials can be materials exhibiting the usual Newtonian behavior. However, non-Newtonian materials should be also expressly included. For example, materials with thixotropic behavior can be used, which in the initial state of the pyrotechnic drive devices are highly viscous and in particular are no longer free-flowing under the effect of simple gravity, but suddenly become much more fluid, or capable of flowing after the effect of increased pressure. However, so-called structurally viscous or pseudoplastic materials can be also used, which are highly viscous in the initial state of the pyrotechnic drive device, but in particular under the simple effect of gravity are no longer free-flowing, and suddenly become more fluid or free-flowing with high shear rates. Common examples of such thixotropic or pseudoplastic materials are tomato ketchup, toothpaste, and many fats or latex adhesive mixtures.

Such substances can in the initial state of the pyrotechnic drive devices still exhibit the desired characteristics, in particular the desired (high) viscosity in order to prevent leaking of the filling material from the combustion chamber during the assembly or during the storage of the device, while after the activation of the drive device, the higher pressure generated in the combustion chamber in this manner results in achieving characteristics that are advantageous for the function of the drive device, in particular the desired lower viscosity.

The filling material may be also a mixture of different materials. In particular, solid particles can be mixed with a liquid or with a gel-like material. A pasty material can thus be produced, whose flow behavior can be adjusted with an admixture of certain solid particles (for example talk, glass beads or highly dispersed silicon, HDK). So for example, an admixture of relatively larger particles improves the flow behavior, while an admixture of many small particles makes as a rule a pasty material more viscous. At the same time, the viscous characteristic is particularly advantageous for storage. Such pasty filing materials often display also the pseudoplastic characteristic described above because after the activation of the pyrotechnic material, the filling material will be moved and the bond between the particles is broken at a certain shear rate, which is to say that the cohesion between the particles and the liquid or gel-like material is lost.

According to one embodiment of the invention, the soft rubber-like material can be a material that is manufactured on the basis of silicon or rubber, which preferably has a hardness of less than 70 Shore A. This makes it possible to ensure that the residual volume is filled substantially completely. A filling body made of such a material can be prefabricated and used together with the pyrotechnic material under pressure in the combustion chamber volume.

If the pyrotechnic material is designed in such a way that a shock wave is generated, which is to say when a detonatively converting material is used, the filling material can be designed or selected in such a way that the shock wave impedance of the filling material essentially corresponds to the impedance of the one or of a plurality of pressure-receiving elements, or so that it differs from it only by a small, predetermined amount. As a result, an optimal coupling of the shock wave generated during the detonative conversion of the pyrotechnic material with the at least one pressure-receiving element is achieved. In particular, relevant reflections on the pressure-receiving surface or surfaces are avoided, which is to say that the reflection factor on the pressure-receiving surfaces essentially equals zero or is below a predetermined acceptable threshold.

The filling material should have, in particular when a detonatively converting material is used, a low sound wave attenuation to make it possible to achieve a transfer of the energy of the shock wave onto the one or multiple pressure-receiving elements with the smallest possible loss amount.

The filling material can be a fluid, in particular an oil. Depending on its desired features, it can be a synthetic or a natural oil, for example a vegetable oil, in particular, sunflower oil can be used. Among natural, vegetable oils, the combination with detonatively converting material has proved in particular excellent when sunflower oil was used as a filling material; with synthetic oils, all thin-liquid silicon oils or silicon olefins are suitable.

According to an embodiment of the invention, the filling material or a component thereof can be a fluid which evaporates completely or partially as a result of the energy released by the pyrotechnic material. This increases the pressure, or generates a pressure wave.

According to one embodiment of the invention, this kind of a fluid or a component thereof can also exhibit a boiling delay, wherein the component with the boiling delay is preferably water. The boiling delay occurs as a result of the rapid heating of the fluid due to pyrotechnically generated energy above the boiling point, whereby a metastable state is reached in which the fluid can evaporate explosively. This increases the generation of pressure waves even more.

A positive side effect of a combustion chamber that is filled with a filling material is that the effect is strongly limited outside in the case when the combustion chamber bursts, because the generation of the desired pressure in the combustion chamber must be limited only to a small fraction of the pyrotechnic mass that would otherwise be required and the fluid, unlike a gaseous medium, can hardly store any energy, which would be released during the bursting of the combustion chamber.

According to another embodiment of the invention, the pressure-receiving element can be a movable piston whose movement path can be limited by a stop means provided in the housing. With this embodiment, the substantially slower pressure increased in the combustion chamber is utilized to generate mechanical output power. Because a movable piston can with its mostly large mass hardly follow a shock wave, it can be moved in the relevant medium with a speed above supersonic speed. The limiting of the movement path of a piston prevents the piston from exiting from the housing of the drive device. The hot gases generated in this manner thus remain in the combustion chamber and they cannot endanger the environment.

According to another embodiment, the pressure-receiving element can be also a membrane, which is held stationary in the housing, or which is held in a movable piston whose movement path is preferably limited by a stop means provided in the housing. The membrane enables a simple transmission of one of the shock waves generated by the detonatively reacting material to one or a plurality of elements to be driven.

The membrane can be provided with an output region, preferably a central region of the membrane, which in the initial state acts upon an element to be accelerated, for example a plunger. In another embodiment, the output region can in the initial state can carry a substance, which adheres to the membrane and which is to be accelerated and separated from it, for example a solid, liquid, or gel-like pharmaceutical substance.

According to one embodiment of the invention, the element or substance to be accelerated can be arranged fully outside of the combustion chamber and a limiting element can be provided, which limits the penetration of the element to be accelerated, or the ejection of the substance to be removed from the membrane, and which also limits the deformation path of the membrane in a region outside of the output region.

According to another embodiment of the invention, the element to be accelerated can pass through the combustion chamber and in the initial state, it can protrude from the combustion chamber or the housing of the pyrotechnical drive device at an opposite position of the combustion chamber, or it is connected flush with the combustion chamber. Also in this case, a limiting element is provided which limits the deformation path of the membrane, preferably in the entire area of the membrane that undergoes deformation during the activation of the pyrotechnic material.

Plastic materials are suitable as materials for the limiting elements, in particular thermoplastics, for example polyoxymethylene (POM), because these materials can be processed inexpensively and their geometry can withstand well and without cracking the violent impacts occurring after the ignition, and because plastic materials can withstand the impacts to which they are subjected so that the stresses on the membrane can be minimized.

A membrane can be designed as a multilayer membrane, preferably as a double-wall membrane having a first and a second wall, which can be connected with an intermediate wall, for example by gluing or bonding.

This provides an increased protection against bursting of the membrane. This intermediate layer can also perform a sealing or gliding function.

Generally, a rapid movement with a small movement path can be decoupled from the drive device by means of a membrane, or an impulse can be transferred to the element to be driven, while a piston is more likely to be used to decouple a slower movement, usually with a larger movement path. A combination of both variants is also possible, when the membrane is provided in a movable piston. The membrane can decouple a rapid movement or a mechanical impulse (which is to say use the pressure shock or the pressure wave) and the piston can used with a slower pressure generation in the combustion chamber, which is practically always present there, for decoupling a slower movement, or simply to increase the volume in the combustion chamber in order to reduce the pressure in the combustion chamber after the decoupling of the rapid movement. Therefore, an excessive deformation of the membrane, or even bursting of the membrane can be avoided, even when the piston movement is not used as such for driving purposes.

In embodiments that used a detonatively converting material, the housing of the combustion chamber, or at least one part of the wall of the combustion chamber, can be made of a material that conducts heat well, such as copper or aluminum. As a result, the rapidly spreading pressure search is still transferred well to the pressure-receiving element, while the slowly developing pressure, whose energy in these embodiments does not contribute to the drive power, is reduced because the thermal energy is absorbed by the housing, or it will be output to the environment through the housing. A filling piece made of a corresponding material can be also provided in the combustion chamber for this purpose, which limits (at least partially) the combustion chamber. In addition, at least one part of the combustion chamber wall can be formed by a filling piece, which consists of a solid material and which from the initial state of the pyrotechnic drive device, which is to say from the state of the pressures and temperatures prevailing in the combustion material, is at least partially liquefied after the activation of the pyrotechnic material, or which is converted into the gaseous state.

This removes energy from the combustion chamber so that the pressure in the combustion chamber is reduced.

Such a filling piece can consist, for example, of dry ice, and it can be designed for lining the entire combustion chamber or a part of the combustion chamber. Dry ice is simple to process and in the final state of the pyrotechnical drive device it generates a relatively low pressure in the combustion chamber, so that a pressure of between approximately 70 and 100 bars is established with the sublimation and the return to the solid state. In certain embodiments, such a relatively low and constant pressure for a long period of time is desirable, for example when a moved piston is used as a pressure-receiving element that is to be maintained in an end position after the activation, or when the deformation of the membrane is to be maintained with a certain force.

According to an embodiment of the invention, the membrane can include performing a directed deformation provided in an area comprising the driving region or within the driving region relating to inward direction, which is designed to deflect of the shock wave and/or to generate a sudden leapfrogging effect.

Other embodiment of the invention are described in the dependent claims.

The invention will now be described in more detail on embodiments thereof with reference to the attached figures. The figures show the following:

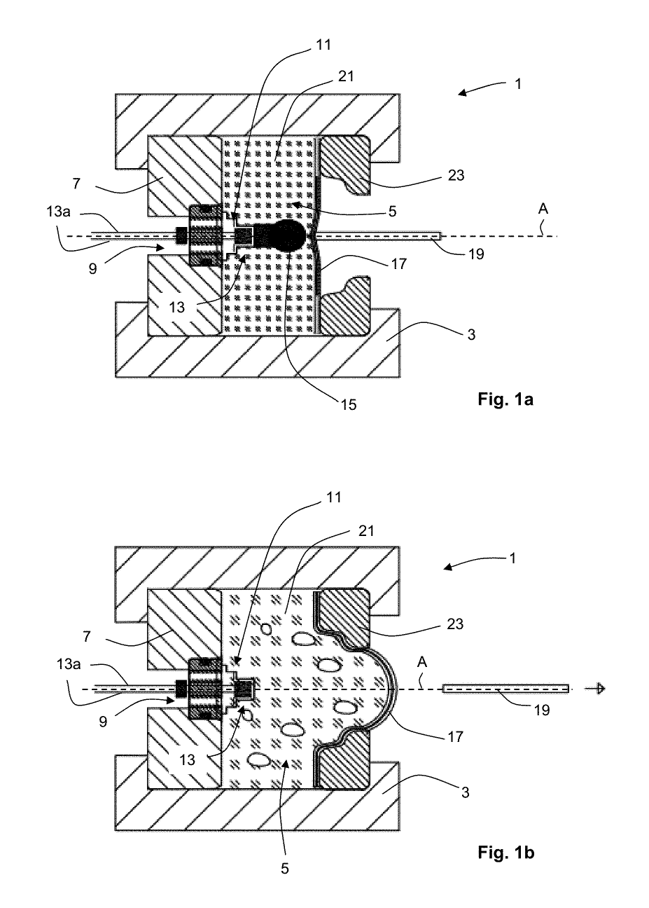

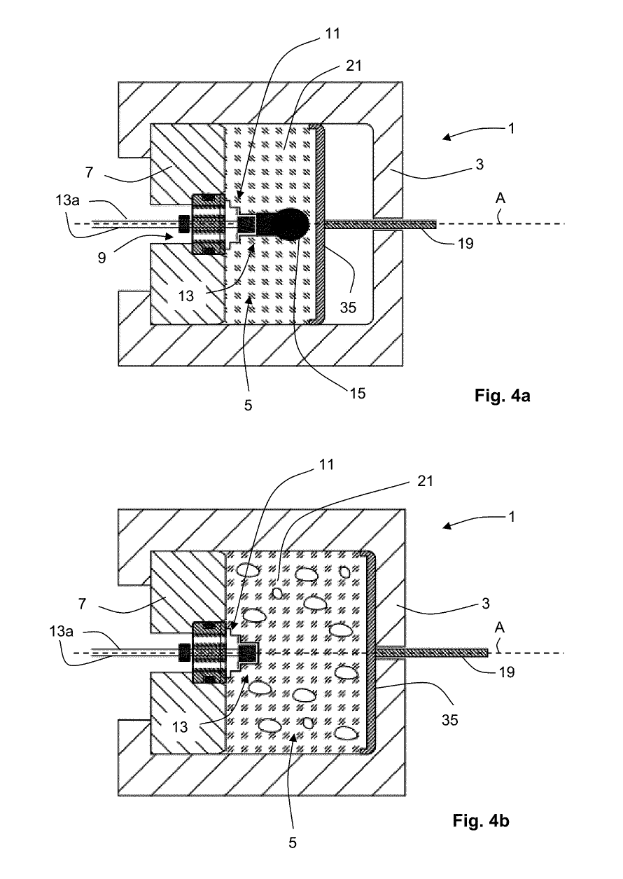

FIG. 1 a schematic longitudinal section of a first embodiment of a pyrotechnic drive device provided with a membrane that impacts with a drive region an element to be driven, wherein in FIG. 1a is illustrated the initial state of the drive device, and FIG. 4b illustrates the state after the activation of the pyrotechnic material and the completion of the driving movement;

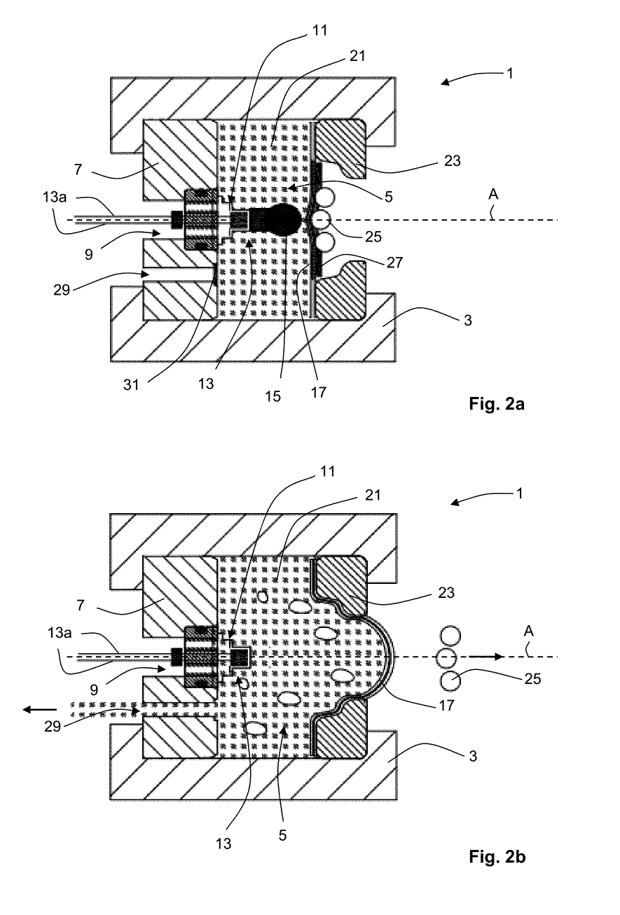

FIG. 2 a schematic, longitudinal section through a second embodiment of a pyrotechnic drive device provided with a membrane that carries dust-like or powder-like particles in a drive region, wherein FIG. 2a illustrates the initial state of the drive device, and FIG. 2b illustrates the state after the activation of the pyrotechnic device and completion of the drive movement and discharging of the dust or powder-like particles;

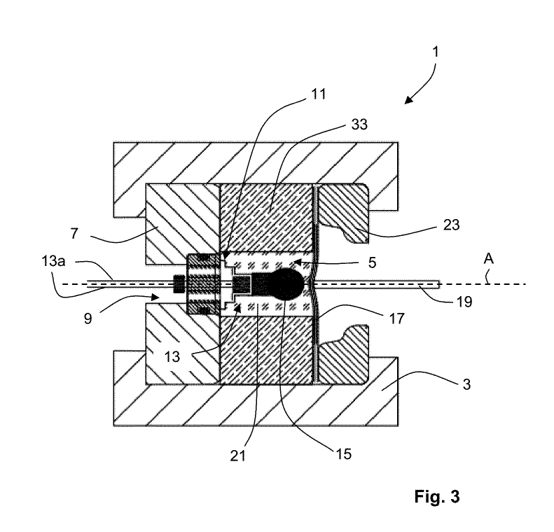

FIG. 3 a schematic, longitudinal section through a third embodiment of a pyrotechnic drive device similar to the variant according to FIG. 1, wherein the combustion volume is reduced by means of an insert part;

FIG. 4 a schematic longitudinal section through a fourth embodiment of a pyrotechnic drive device provided with a movable piston, which is connected with a pin-like drive element, wherein FIG. 4a illustrates the initial state of the drive device, and FIG. 4b illustrates the state after the activation of the pyrotechnic material and completion of the drive movement;

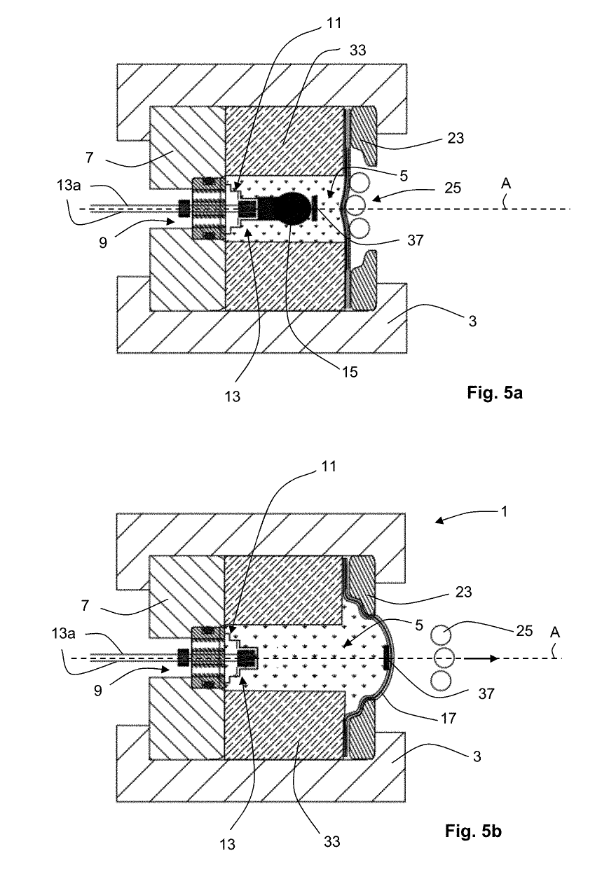

FIG. 5 a schematic longitudinal section through a fifth embodiment of a pyrotechnic drive device similar to the variant shown in FIG. 3 provided with an impulse transmission element in the combustion chamber, wherein FIG. 5a illustrates the initial state of the drive device, and FIG. 5b illustrates the state after the activation of the pyrotechnic material and completion of the drive movement.

FIG. 1 shows a first embodiment of a drive device 1, which is designed as an independent, functional device. It goes without saying that such a device can be also integrated in a superordinate device, for example a pyrotechnic injection device without a needle, or an electrical limit switch. In this case, one or several elements of the pyrotechnic drive device 1 are designed so that they are integrated with a corresponding superordinate device. The pyrotechnic drive device 1 comprises a housing 3 in which is provided a combustion chamber 5. The combustion chamber 5 is limited on a rear side by a floor element 7, which is provided with a receiving recess 9 arranged on the longitudinal axis of the pyrotechnic drive device 1, in which is inserted a pyrotechnic unit 11 provided with a foot region. The pyrotechnic unit 11 comprises an ignition device 13, which is connected to pyrotechnic material 15. At least the pyrotechnic material 15 is projecting from the floor element 7 into the combustion chamber 5.

The pyrotechnic material 15 can be a pyrotechnic substance with a deflagrating or detonative conversion that is preferably formed in one body with a predetermined geometry, or that is at least formed with an outer skin, preferably flexible skin, delimiting the rest of the combustion chamber volume on the opposite side. The pyrotechnic material 15 can be for example formed from a powder that is pressed into a shaped body, which is additionally surrounded by a flexible, for example rubber-like, outer skin. The outer skin can also serve for connecting the pyrotechnic material 15 or the relevant shaped body with the ignition device 13.

The front part of the end region of the combustion chamber 5 is delimited by a membrane 17, which is plastically and/or elastically deformable at least in the central region with respect to the longitudinal axis A. The membrane 17 can be manufactured as shown in FIG. 1a which shows the initial state of the pyrotechnic drive device 1 prior to an activation of the pyrotechnic unit 11, so that the membrane 17 is provided with a centric form that is directed inwards with respect to the combustion chamber 5, which is referred to as pre-forming and, depending on the material of the membrane 17, either with cold or warm deformation of a membrane that is initially planar. This pre-formation, which includes the output region of the element 19 to be driven, can contribute with the activation of the pyrotechnic unit 11 to amplification of the impulse or the energy that is to be transmitted to the element 19 to be driven or accelerated. The pre-punching of the membrane 17 can initially work against a deformation of the membrane when the pressure is initially increased inside the combustion chamber 5 and then it can cause a sudden “leapfrogging” from the stable state that is present in the initial state of the pyrotechnic drive device 1, which is then quickly shifted to another, stable or unstable state. This second state, however, is of little relevance because the membrane 17 will be in any case deformed with the activation of the pyrotechnic unit 11 in the end state (FIG. 11b). A similar pre-forming of the membrane 17 can also cause a deflection or focusing of the shock wave (hereinafter referred to as shock wave deflection) onto the output area of the membrane. With the leapfrog effect or with the focusing of the shock wave, an additional increase of the achievable surface velocity of the membrane in the output region of about 10 to 20 percent has been proven with measuring technology.

The residual volume of the combustion chamber 5 that is not claimed by the pyrotechnic material 15 or by the ignition device 13, which is to say by the part of the pyrotechnic unit 11 protruding into the ignition chamber, is filled essentially completely with a filling material 21. The filling material can be designed as a liquid or a gel-like material. A soft, rubber-like filling material or a combination of such a soft, rubber-like filling material with another liquid of gel-like material is also possible. In any case, however, a substantially complete filling of the residual volume of the combustion chamber 5 should be ensured.

When the pyrotechnic drive device 1 is activated from the initial state illustrated by FIG. 1, the process of the conversion of the pyrotechnic material 15 is started by means of the ignition device 13. The ignition device can be in this case for example an electric ignition device that can be controlled in a corresponding manner with electrical connections 13a. Ignition devices of this type are known in various forms and thus need not be described in more detail here. Instead of electrically controllable ignition devices, other ignition devices can be of course also used, for example devices that can be triggered with shocks, which is to say with mechanical accelerations.

With the conversion of the pyrotechnic material 15, the pyrotechnic drive device 1 shown in FIG. 1 is converted to the end state illustrated by FIG. 1. In this case, two different mechanisms determine the resulting generation of an output performance in a driven central region of the membrane 17 that is centered around the longitudinal axis A.

On the one hand, pyrotechnic material 15 with a deflagrating conversion can be used, which generates gas pressure in the combustion chamber 5, wherein the pressure increase is slower in the filling material 21 than the velocity of sound. In this case, the membrane is deformed until an initial state corresponding to FIG. 1b is reached.

On the other hand, a pyrotechnic material with a detonative conversion can be also used, which at first generates a shock wave in the combustion chamber 5, which then spreads faster than at the velocity of sound in the filling material 21. This shock wave is first transferred to the membrane 17 and then from the membrane to the element 19 to be driven, which in the initial state rests against the membrane 17 in the output region. This leads to a transmission of a high impulse to the element 19 to be driven, which is then thrown away from the membrane surface with a corresponding amount of kinetic energy (see FIG. 1b).

In addition, the detonatively converted pyrotechnical material 15 also leads to a slower increase of the pressure in the combustion chamber 17, so that a deformation of the membrane takes place also in this case. However, this effect occurs only after the energy of the shock wave, which impacts the output region of the membrane 19, has already been transferred to the element 19 to be driven.

In order to limit the deformation of the membrane 17 in the final state illustrated by FIG. 1b, an annular limiting element is used that is also held in a coaxial position to the longitudinal axis A in the housing 3. For this purpose, the housing can be provided on its front side with a flange that surrounds the annular boundary element 23 radially inwardly on its front side.

The mounting of the floor element in the housing 3 can be carried out in a similar manner. The housing can for this purpose be provided with a bottom wall that is directed radially inward. The floor element 7 is supported in the axial direction against the inner side of the bottom wall.

In order to mount the pyrotechnic drive device 1, the floor element 7 can be first pushed into the housing with the pyrotechnic unit already arranged inside until the floor element 7 rests with its lower side against the rear side on the bottom wall of the housing 3. After that, the filling material can be introduced inside. At the end, the membrane 17 and the boundary element 23 are inserted into the cylindrical housing 3, which is still open in the front, so that the membrane rests with its inner side on the filling material. After that, a mechanical flanging of the cylindrical housing wall is performed in such a way that the boundary element is safely held in the housing and the membrane is pressed with a predefined force against the filling material 21. This makes it possible to avoid creating cavities in the combustion chamber that are not filled with the filling material 21.

During the conversion of the pyrotechnic material 15, in addition to generating gas pressure, which itself results from the process of the conversion of the pyrotechnic material 15, additional gas pressure can be also generated in this manner when at least some of the filling material 21 is converted into the gaseous state by the energy released during the transfer process. The mass of the pyrotechnic material can thus be reduced relative to the initial state by using only a combustion chamber that is filled with gas. In addition, a steeper pressure increase can be achieved in this manner so that the membrane is converted faster, which is to say with a higher acceleration, from its initial state into the final state. If the element 19 to be driven is accelerated essentially only by using the deformation movement of the membrane 17, then an additional conversion of the filling material 21 in the gaseous state in this manner is advantageous. The filling material 21 can be therefore selected appropriately while taking into account this requirement.

Particularly suitable for this purpose are liquids such as natural oils, in particular vegetable oils.

On the other hand, if the energy of a shock wave is supposed to be transmitted to the element 19 to be driven, then the conversion of the filling material 21 in the gaseous state is rather undesirable. This is because the energy of the shock wave will be transferred to the element 19 to be driven at a point in time prior to the point in time of the slow deformation of the membrane, which is caused by the generation of the hot gases in the combustion chamber 5.

When ejection of one or more elements 19 to be driven from the output region of the membrane 17 is desired, the use of a detonatively converting pyrotechnical material 15 is in particular desirable when one or a plurality of elements 19 to be driven have a relatively small mass and when they should be ejected with as high velocity as possible.

On the other hand, pyrotechnic material with a deflagrating conversion should be used in the case when one or a plurality of elements 19 have a higher mass, or when they should be ejected with a smaller velocity from the surface of the membrane 17.

In addition, the latter moving mechanism should be used above all when the element 19 to be driven is firmly connected with the membrane 17 and with other elements or other devices to be impacted.

The other embodiment of the pyrotechnic drive device 1 illustrated in FIG. 2 differs from the embodiment indicated in FIG. 1 only in the features described below, so that the features identical to those of the embodiment shown in FIG. 1 do not need to be described again.

In the embodiment illustrated in FIG. 2, an adhesive layer 27 is provided on the membrane 17 in the central output region instead of an element 19 to be driven, which serves to fix a powder-like or dust-like substance 25. Only three oversized particles symbolizing the dust-like or powder-like substance are shown in FIG. 2. The adhesive layer can be for example an adhesive coating or a dried sugar solution. The powder-like or dust-like substance can be a medication for injection into human or animal issue.

In this type of an embodiment, in accordance with the embodiment above, a detonatively converting material 15 is principally used because the powder and dust-like particles of the substance 25 must be accelerated to as high velocity as possible so that they will penetrate the surface of the tissue into which injecting should take place to a sufficient depth.

FIG. 2b shows the final state of the pyrotechnic device and of the powder or dust-like substance particles of the substance 25 that have been already ejected from the surface of the membrane 17. Since parts of the adhesive layer 27 can also be detached from the membrane surface, the adhesive layer should consist of a material or substance that has a correspondingly neutral conduct, or at least does not lead to adverse consequences.

As shown in FIGS. 1 and 2, the membrane 17 can be formed with multiple layers. In particular, a first and a second layer can be connected via a connecting layer to a total membrane 17. This drastically reduces the likelihood of a bursting of the membrane since it is considered extremely unlikely that both membrane layers would have faulty weak points in the same location, which would lead to the destruction of the layer in question in the absence of the second layer. Moreover, additional stability can be achieved with the connecting layer.

The pyrotechnic drive device 1 shown in FIG. 2 is provided in the floor element 7 additionally with a gas outlet opening 29 that is in the initial state closed with a membrane 31. The membrane 31 is in this case designed in such a way that it will be destroyed when a certain limiting pressure is exceeded in the combustion chamber.

Since a shock wave that is generated by a detonatively converting pyrotechnic material 15 is used in the embodiment shown in FIG. 2 in order to accelerate the substance particles of the substance 25, rather than using the relatively slow deformation of the membrane 17 resulting from the gas pressure that is built up (relatively slowly) in the combustion chamber 5, a weaker membrane 17 can be in this case formed when the gas pressure occurring inside the combustion chamber 5 is limited to a relatively small maximum value. This is determined by the geometry of the gas outlet opening 29 and by the limiting pressure at which the membrane 31 will be destroyed.

As one can see from FIGS. 1 and 2, the task of the limiting element 23 is to limit the deformation of the membrane 17 and to support the membrane in a annular region that surrounds the output region as soon as the deformation of the membrane 17 has progressed so far that the relevant membrane regions fit tightly against the inner wall of the limiting element 23.

Another embodiment, illustrated in FIG. 3, corresponds for the most part to the embodiment shown in FIG. 1. It differs only in that the volume of the combustion chamber 5 is reduced by an annular filling piece 33. The geometry of the filling piece 33 can in this case be selected in such a way that that the combustion chamber 5 is kept as small as possible. However, the front region of the combustion chamber 5 must be designed in the cross-section (perpendicular to the longitudinal axis A) at least in such a way that this cross-section is as large as the output region of the membrane, which contributes to the transfer of the energy of the shock wave, or through its deformation causes the transfer of energy onto at least one element 19 to be driven.

As mentioned above, the filling piece 33 can also consist of a material that is solid in the initial state of the drive device 1 and that is converted with the increase of the pressure and temperature in the combustion chamber after the activation of the pyrotechnic material into fluid or gaseous state. In particular, the filling piece 33 can also consist of dry ice, which is easily processed and in the final state of the drive device in the (closed) combustion chamber generates a constant pressure, with which the membrane 17 is constantly impacted. An element to be driven that is connected with the membrane or permanently impacted can thus be kept in its end position.

It should be at this point mentioned that the element 19 to be driven is obviously not only acted upon or connected to the outward facing side of the membrane 17, relative to the combustion chamber, or that it can be connected with this side, as illustrated in FIGS. 1, 3 and 4. Rather, the element 19 can be also impacted by the inner side of the membrane, relative to the combustion chamber 5, or it can be connected to it. In this case, the element 19 to be driven penetrates through the combustion chamber 19 and it can with its end region protrude from the combustion chamber and possibly also from the housing 3 of the pyrotechnic drive device 1 in question. In this case, the element 19 to be driven should be preferably connected with the membrane, or it should be designed integrally with the membrane. At this point, when the element 19 to be driven leaves the combustion chamber and for example protrudes through a corresponding opening in the floor part from floor part 7, the element 19 to be driven can be sealed with a sealing means opposite the interior of the combustion chamber, or a through-passage opening in the floor part 7. However, if a gel-like, a pasty or a rubber-like filling material is used in the interior of the combustion chamber 5, the sealing can be omitted if it is acceptable in the relevant application that hot gases can flow out of an outlet opening that is not sealed, for example in a circular region surrounding the element to be driven, and exit from the housing 3 or from the floor part 7.

However, the remaining unsealed outlet opening may be only so large that a pressure that is sufficient for deformation of the membrane can still be built up in the interior of the combustion chamber. The limiting element 23 can be in these embodiments designed in such a way that the entire outer side of the membrane is supported in the end state. This is because the limiting element 23 is in this case not be clamped down by an element to be driven.

In this embodiment, the pyrotechnic unit 11 can be of course designed also so that it is not provided in the axis A of the pyrotechnic drive device 1. Since the positions of the pyrotechnic material 15 or of the ignition device 13 are not critical, the pyrotechnic unit 11 can be projecting in this embodiment from one side of the housing 3 into the combustion chamber. At the same time, the pyrotechnic material 11 must be in any case provided in such a way that the it does not interfere with the movement of the element 19 to be driven, which passes through the combustion chamber 5.

The embodiment of the a pyrotechnic drive unit 1 illustrated by FIG. 4 differs from all the embodiments described previously in that a displaceable piston 35 is used instead of a membrane, which transfers the energy to one or to a plurality of the elements 19 to be driven, or to substance particles adhering to a surface so that the piston delimits the combustion chamber 5 (FIG. 4a). The housing 3 is in this case designed on its front side in such a way that only a relatively small through-opening is provided in the longitudinal axis A of the pyrotechnic unit 1, thorough which an element 19 to be driven is projecting. The element 19 to be driven can be connected with the piston 35, as is the case shown in the illustration.

Otherwise, the pyrotechnic drive unit illustrated in FIG. 4 is provided with the same features as those of the embodiment shown in FIG. 1, in particular with respect to the housing, the bottom part and the pyrotechnic unit.

The piston 35 can be fixed in its initial position in the housing 3, for example with a latching means, which is provided on the inner wall of the housing and/on the outer wall of the piston 35. This latching means or fixing means ensures that the piston 35 will be accelerated only when a limiting pressure is exceeded in the direction of the front side of the housing 3.

In this embodiment, a pyrotechnic material 15 with a deflagrating conversion is preferably used because the piston 35 with its carried mass cannot really be moved by an impact wave alone. That is why the gas pressure that is built up in the volume of the combustion chamber is required for this purpose.

As shown in FIG. 4, a sealing effect can be achieved on the outer circumference of the piston 35 so that a relatively thin circumferential wall region of the piston can be impacted in the radial direction of the gas pressure built up in the combustion chamber. A radial pressure of the circumferential, relatively thin wall onto the inner wall of the house is achieved in this manner. This results in the desired sealing effect during the buildup of the gas pressure in the combustion chamber also during the movement of the piston 35 in the direction of the axis A.

As one can see from FIG. 4b, the element 19 to be driven is in the embodiment shown in FIG. 4 connected with the piston 35 in a fixed manner. The element 19 can thus serve as an output element impacting another element or another device. However, it goes without saying that the element 19 to be driven can be also designed so that it is not connected with the piston 35. In this case, it can be impacted already in the initial state by the piston 35. The fixing of the element 19 can be carried out in this case with a corresponding design of the through opening in the housing 3. However, the element 19 cannot be impacted by the piston 35 also in the initial state and it is fixed only in the through-opening of the housing 3. The piston 35 in this case impact first with a certain velocity the element 19 to be driven and transfers a mechanical impulse to the element 19, so that it will be accelerated in the direction of the axis A and thrown away.

Also in the embodiment illustrated in FIG. 4, the element 19 to be driven can in one variant pass through the combustion chamber. Reference is made to the preceding embodiment with respect to this variant.

The embodiment of a pyrotechnic drive device 1 illustrated in FIG. 5 corresponds to a combination of the embodiments of FIGS. 2 and 3. A membrane is used also here, in which a powder-like or dust-like substance is applied to the outer surface of the output region of the membrane 17. This adhering layer is omitted here to suggest that pure adhesion can be also sufficient when a powder-like or dust-like substance 25 is provided on the surface of the membrane 17. However, an adhering layer, for instance oil or sugar solution, can be of course also used here. Other than that, the embodiment shown in FIG. 5 substantially corresponds to the embodiment shown in FIG. 2, except that a filling piece 33 corresponding to the embodiment according to FIG. 3 is provided in the combustion chamber.

In addition, the functionality of the embodiment according to FIG. 5 differs from the preceding embodiments in which a detonatively converting pyrotechnic material 15 was used in that an impulse transmission element 37 is provided in the direction of axis A in front of the pyrotechnic material 15. This impulse transmission element 37 is designed with such a geometry with respect to the material and mass that it is capable of being accelerated with the generated pressure shock or with the generated pressure wave in a very short time period to such a velocity that the impulse transmission element almost rides, to put it in this manner, on the front of the shock wave. This makes it possible to achieve that the impulse transmission element will be thrown essentially together with the foremost front of the shock wave against the inner wall of the membrane 17. This means that not only the energy contained in the shock wave, but also the mechanical impulse of the impulse transmission element 37 is used for transmitting an impulse by means of a very rapid deformation of the membrane 17 in the output region in order transmit at least one component thereof to the membrane 17 and via the membrane 17 to the particle of the substance 25.

The end position of the membrane 17 and also of the impulse transmitting element 37 is illustrated in FIG. 5b. In the end position, the deformation of the membrane 17, (which cannot be avoided and which is not contributing to the acceleration of the substance particles), is again evident.

In the embodiments according to FIGS. 3 and 5, the filling piece 33 is used to ensure fixing of a circular edge region of the membrane 17, so that this end region is clamped between the filling piece 33 and the limiting element 23. However, the membrane 17 can of course also be connected with the relevant edge region with the filling piece 33 or with the limiting element 23.

The filling piece can be also designed with respect to its geometry so that not only a reduction that is as large as possible of the volume of the combustion chamber will be achieved, but so that focusing of shock wave generated by the pyrotechnic unit 11 on the output region of the membrane will be also achieved. For this purpose, the axial through-opening in the filling piece 33 can be formed with a conically extending shape in the direction of the membrane.

The embodiment described above can be obviously also modified to create other variants, wherein certain features of some of the embodiments are combined, when this is appropriate, with the features of some of the other embodiments.

All embodiments of the pyrotechnic drive unit can be either integrated in a superordinate device, for example an injection device without a needle, or an electrical switch or the like, or they can be designed in the form of an independent unit.

### LIST OF REFERENCE SYMBOLS

1 pyrotechnic drive device
3 housing
5 combustion chamber
7 floor element
9 receiving recess
11 pyrotechnic unit
13 ignition device
13a electrical connections
15 pyrotechnic material
17 membrane
19 element to be driven
21 filling material
23 limiting element
25 powder-like or dust-like substance
27 adhering layer
29 gas outlet opening
31 membrane
33 filling piece
35 piston
37 impulse transmission element
A longitudinal axis

What is claimed is:

1. A pyrotechnic drive device comprising:
a housing (3), in which a combustion chamber (5) is arranged with a pyrotechnic material (15),
an activation device (13) arranged in the combustion chamber (5) for activating the pyrotechnic material (15),
wherein the combustion chamber (5) is delimited in at least one state of the pyrotechnic drive device by combustion chamber walls (3, 7, 13, 17, 33, 35), closed on all sides, which are formed in one or a plurality of partial regions each time by a respective pressure-receiving surface with a pressure-receiving element (17, 35),
wherein each pressure-receiving surface of each pressure-receiving element (17, 35) is impacted after the activation of the pyrotechnic material (15) in such a way by the pressure generated in this manner that the pressure-receiving element (17, 35) is moved and/or deformed and/or a mechanical impulse is transmitted via the pressure-receiving element (17, 35) to an element to be driven (19) that is connected to it mechanically at least in the initial state, or wherein it is transmitted to a substance (25) that is connected to it, and
wherein a residual volume of the combustion chamber (5), in which no pyrotechnic material (15) is provided in the initial state, is substantially filled with a liquid, gel-like or pasty filling material (21) and/or a soft rubber-like filling material (21).

2. A pyrotechnic drive device according to claim 1, wherein the pyrotechnic material (15) is a detonatively converting material or a deflagratingly converting material.

3. A pyrotechnic drive device according to claim 1, wherein the soft rubber-like filling material (21) is manufactured as a silicon-based material, or a rubber-based material, which preferably has a hardness that is smaller to or equal to 70 Shore A hardness.

4. A pyrotechnic drive device according to claim 1, wherein the pyrotechnic material (15) is designed in such a way that a shock wave is generated and the filling material (21) is obtained so that the shock wave impedance of the filling material (21) substantially corresponds to the shock wave impedance of one or of the plurality of pressure receiving elements (17, 35), or differs from it only by a predetermined small amount.

5. A pyrotechnic drive device according to claim 1, wherein the filling material (21) has a low shock wave attenuation so as to achieve of the energy of the shock wave, which is generated during the activation of the pyrotechnic material (15), to one or a plurality of pressure-receiving elements (17, 35) with a loss that is as small as possible.

6. A pyrotechnic drive device according to claim 1, wherein the filling material (21) is a synthetic oil, for example silicon oil, or a natural oil, for example a vegetable oil, in particular sunflower oil, or silicone oil gel.

7. A pyrotechnic drive device according to claim 1, wherein the filling material or a component part thereof has a boiling point, wherein the boiling point of the component part is preferably the boiling point of water.

8. A pyrotechnic drive device according to claim 1, wherein the pressure-receiving element is a movable piston (35), whose path of movement is preferably limited by a stop means provided in the housing (3).

9. A pyrotechnic drive device according to claim 1, wherein the pressure-receiving element is a membrane (17), which is held in a stationary manner in the housing (3), or which is held in a movable piston, and whose movement path is preferably limited by a stop means provided in the housing (3).

10. A pyrotechnic drive device according to claim 9, wherein the membrane (17) is provided with an output region, which is preferably a central region of the membrane (17), wherein in the initial state, an element (19) to be driven is impacted, for example a plunger, and the membrane (17) is provided with an output region, which is preferably a central region of the membrane (17), which carries in the initial state a substance (25) adhering or connected to the membrane (17) in the initial state, which is to be detached, for example a pharmaceutical substance.

11. A pyrotechnic drive device according to claim 10, wherein the element (19) to be driven or the substance (25) is arranged completely outside of the combustion chamber (5) and that a limiting element (23) is provided with a through opening for the element (19) to be driven or for the ejection of the substance (25) to be ejected from the membrane (17) and which delimits the deformation path of the membrane (17) in a region outside of the output region.

12. A pyrotechnic drive device according to claim 10, wherein the element (19) to be driven passes through the combustion chamber (5) and is arranged in the initial state in a position of the combustion chamber (5) facing away from the membrane (17), or projects from the combustion chamber (5) or from the housing (3) of the pyrotechnic drive device (1), or is connected flush with it, wherein a limiting element (23) is preferably provided, which delimits the deformation path of the membrane (17), preferably in the entire region of the membrane (17), and which undergoes a deformation with the activation of the pyrotechnic material (15).

13. A pyrotechnic drive device according to claim 9, wherein the membrane (17) is designed as a multi-layered membrane, preferably as a membrane with a double wall having a first and a second wall, wherein the walls are connected via an intermediate wall, for example by gluing.

14. A pyrotechnic drive device according to claim 1, wherein the pyrotechnic material (15) is a detonatively converting material, and the housing (3) of the combustion chamber (5) or at least a part of the combustion chamber wall consists of a material which conducts heat well, for example a metal that conducts heat well, such as copper or aluminum, and that at least a part of the combustion chamber wall is formed with a filling piece (33), which consists in the initial state of the pyrotechnic drive device of a solid material that is converted after the activation of the pyrotechnic material (15) at least partially into the liquid or gaseous state.

15. A pyrotechnic drive device according to claim 1, wherein the pyrotechnic drive device is designed as a functional unit for installation or interchangeable insertion into a superordinate device, for example a pyrotechnic electronic switch, or a pyrotechnic injection device without a needle for injecting a dust-like or powder-like substance into a body tissue.

16. A pyrotechnic drive device according to claim 9, wherein the membrane (17) is provided with a pre-formed region comprising an output region, or provided within an output region, directed inward with respect to the combustion chamber (5), which is designed for deflecting a shock wave and/or for generating a leapfrog effect.
